(19) United States
(12) Patent Application Publication
(10) Pub. No.: US 2006/0217660 A1
(43) Pub. Date: Sep. 28, 2006

(54) Catheter with semipermeable membrane for treatment of severe pulmonary emboli

(76) Inventors: Banning Gray Lary, Miami, FL (US); Todd Pendleton Lary, Miami, FL (US)

Correspondence Address: MCHALE & SLAVIN, P.A., 2855 PGA BLVD, PALM BEACH GARDENS, FL, 33410, US

(21) Appl. No.: 11/336,401

(22) Filed: Jan. 20, 2006

Related U.S. Application Data
(63) Continuation-in-part of application No. 11/200,766, filed on Aug. 8, 2005.
(60) Provisional application No. 60/661,528, filed on Mar. 10, 2005.
(60) Provisional application No. 60/690,177, filed on Jun. 9, 2005.

(51) Int. Cl. A61M 29/00

(52) U.S. Cl. 604/96.01

(57) ABSTRACT

A catheter system for treating pulmonary emboli has a multi-lumen primary catheter for accessing the vascular system. The primary catheter has a balloon on the distal end for occluding the venous tree, preferably distal to the renal veins. A semipermeable membrane is attached to the primary catheter intermediate the balloon and the proximal end of the primary catheter. Oxygen is supplied to the membrane through the primary catheter to enrich the blood in the venous tree flowing to the heart. Other lumen in the primary catheter guide other catheters through the heart to the pulmonary arteries to inject lysing agents, contrast media, medicaments and to remove blood clots and other catheters may extend through the vena cava to other parts of the venous tree to supply the same agents and to remove blood clots there.

### RELATED APPLICATIONS

This application is a continuation in part of U.S. patent application Ser. No. 11/200,766, filed Aug. 8, 2005, for Improved Catheter for Treatment of Severe Pulmonary Emboli, for which priority of filing date is claimed from U.S. Provisional Application 60/661,528 filed Mar. 10, 2005 and 60/690,177 filed Jun. 9, 2005. The content of each of the prior applications is incorporated herein by reference.

### FIELD OF THE INVENTION

This invention relates to the apparatus and methods of endovascular treatment of blood clots obstructing passageways in the circulatory system.

### BACKGROUND OF THE INVENTION

Thromboembolism is a serious and life threatening problem. The emboli can be sudden and massive and at other times they may be small and multiple. They can be any size and happen at any time.

When blood clots form in the venous circulation of

When blood clots form in the venous circulation of the body they may move or embolize to the lungs. The clots typically embolize from the veins of the legs, pelvis, or inferior vena cava to the right heart cavities and thence into the pulmonary arteries. This results in right heart failure and decreased blood flow through the lungs with subsequent decreased oxygenation of the lungs, heart and the rest of the body. When clots enter the pulmonary arteries obstruction and spasm of the different arteries of the lung occurs which further decreases blood flow and gaseous exchange through the lung tissue resulting in pulmonary edema. All of these factors decrease the oxygen in the blood in the left heart. The oxygenated blood supplied by the coronary arteries to the musculature of both the left and right heart is insufficient for proper contractions of the muscle which further decreases the entire oxygenated blood flow to the rest of the body.

This malady is common and has many causes, among them are prolonged inactivity such as bed rest, dehydration, extensive surgery or protracted disease and many others in which the blood of the inferior peripheral major circulatory system may coagulate to varying degrees with permanent drainage problems.

### DESCRIPTION OF THE PRIOR ART

The prior art contains numerous treatments for this malady, including anticoagulants, antibiotics, peripheral constrictive bandages and surgical attempts at removal of the emboli from the pulmonary artery. The surgical attempts may rely on catherterization of the affected vessels and application of chemical or mechanical agents or both to disintegrate the clot. For example, U.S. Pat. No. 6,852,097 to Fulton III discloses using mechanical agitation and chemical agents to destroy blood clots.

### SUMMARY OF THE INVENTION

What is lacking in the art is an apparatus for maintaining blood oxygenation levels in the circulation system until the emboli can be eliminated and normalized blood flow re-established.

Accordingly, it is a primary objective of the instant invention to provide a catheter system for removing clots and oxygenating the blood using a multi-lumen catheter with a semipermeable membrane.

It is a further objective of the instant invention to prevent venous clots from approaching the heart.

It is yet another objective of the instant invention provide a semipermeable exchange membrane for introducing oxygen into the circulation system.

It is a still further objective of the instant invention to provide a port for introducing the catheter system into the circulation system.

It is another objective of the instant invention to provide cumulative treatment of other portions of the circulation system.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a simplified cross section of the circulatory system with the catheter system of this invention inserted therein.

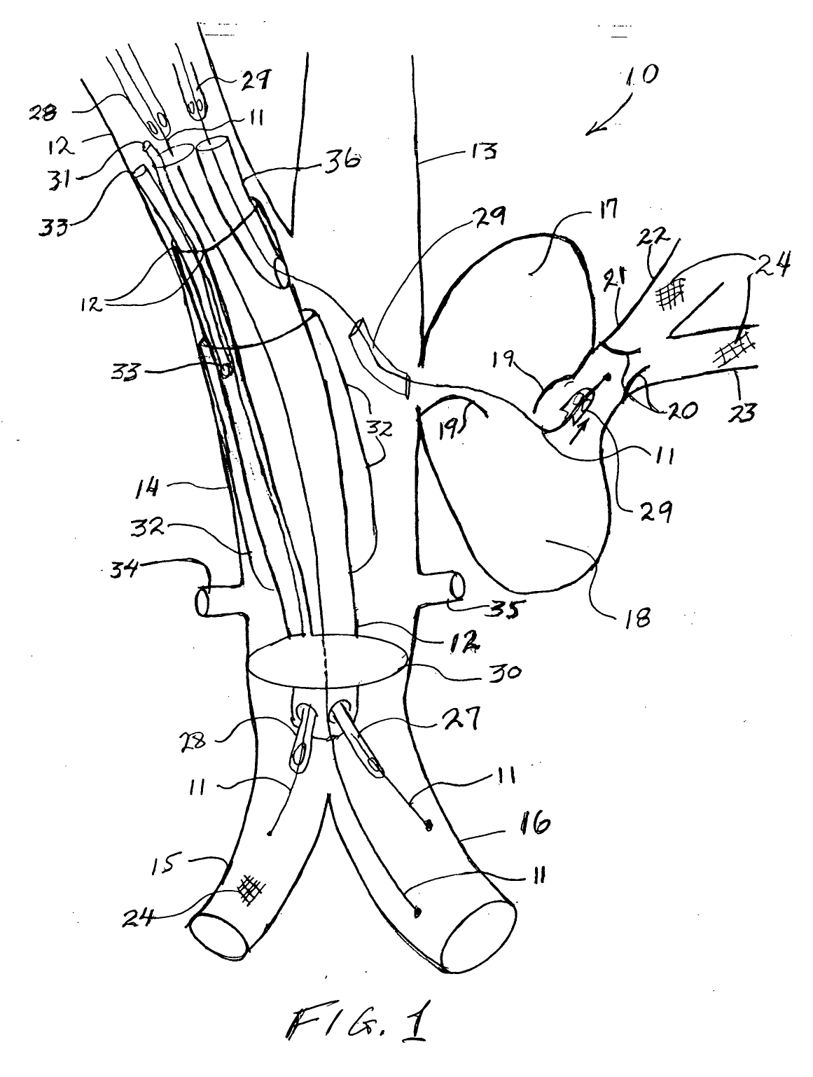

### DETAILED DESCRIPTION OF THE INVENTION

When the diagnosis of acute severe pulmonary embolism is established, the apparatus 10 is applied as rapidly as possible. Using a local anesthetic a small incision is made over the external jugular vein, or internal jugular or similar vein. Blood clots 24 may be in any part of the venous tree and the pulmonary arteries 22 and 23.

As shown, a guide wire 11 is inserted into the right external jugular vein 12 and passed inferior in the vena cava past the entrance of the right renal vein 34 and left renal vein 35. Radiographic utilities are used where possible for visualization of the placement of the guide wire. Contrast media may be used to demonstrate the location of the clot(s).

Over the guide wire 11 is passed a primary multi-lumen balloon catheter 12. The catheter 12 follows the guide wire until the distal end passes the renal veins. The occluding balloon 30 is distended via lumen 31 distal to the renal veins in the venous tree proximal to the clot(s). The inflated balloon prevents further emboli and decreases central venous hypertension to immediately improve right heart failure. The various lumen of the primary catheter provide pathways for introduction of suction and maceration devices to reduce and remove an embolism. Also, chemical agents may be transported to dissolve the clot.

A semipermeable membrane 32 is attached to the catheter 12 intermediate the balloon 30 and the outlet opening for the pulmonary catheter 36. The semipermeable membrane 32 encircles the catheter 12 in the form of a pouch or balloon defining a space between the catheter and the membrane. Oxygen is introduced into the interior of the pouch by lumen 33 in catheter 12. Wetting agents and anticoagulants may also be introduced through lumen 33. The semipermeable membrane 32 permits a superfine mist or fog of oxygen in ionic form to pass into the vena cava along with the other agents, if supplied. The oxygen is taken up by the oxygen deprived blood that is returning to the right auricle 17 of the heart.

An additional multi-lumen catheter 29 may be passed through lumen 36 over previously placed guide wire 11 to enter the right auricle 17, the right ventricle 18 and the pulmonary arteries 22 and 23 through the bicuspid valve 19 and the pulmonary valve 20. This catheter 29 may have multi-lumen for maceration, lysis and suction removal of pulmonary clot(s).

Additional guide wires 11 and multi-lumen catheters 27 and 28 may be passed through lumen in primary catheter 12 and exit distal to the occluding balloon 30 to deliver chemical and/or mechanical agents to clots such as the right iliac vein 15 and left iliac vein 16. The deployment and use of catheter 27, catheter 28 and catheter 29 may be simultaneous or sequential but form part of the treatment regimen.

As soon as practical, oxygen is supplied through the semipermeable membrane 32 to the venous blood proximal to the distended balloon to improve the systemic blood oxygenation in the vena cava. The semipermeable membrane is inflated with minimal pressure to sustain an oxygen rich environment.

The semipermeable membrane 32 permits the circulation of gaseous oxygen through the membrane resulting in an exchange of oxygen and carbon dioxide with an increase of oxygen saturation of the venous blood returning to the heart through the right auricle, right ventricle, the pulmonary arteries and their branches. This, in turn, would decrease pulmonary edema and permit greater oxygenation through the normal alveolae into the blood that enters the left ventricle and subsequently to the coronary arteries which improves the cardiac contractions and increases the delivery of increased oxygenated blood to the rest of the body.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

I (We) claim:

1. A catheter system for endovascular procedures comprising a primary catheter for accessing the vascular system from outside the patient, said primary catheter having a plurality of lumen extending from the proximal end through the distal end, a semipermeable membrane surrounding said primary catheter, said membrane forming a pouch having an open interior, said membrane connected to said primary catheter intermediate said proximal end and said distal end, one of said plurality of lumen in said primary catheter adapted to supply oxygen to said interior of said pouch, said semipermeable membrane adapted to pass minute quantities of oxygen to the vascular system.

2. A catheter system for endovascular procedures of claim 1 wherein said primary catheter includes a pulmonary lumen, said pulmonary lumen extending from said proximal end to an opening in said primary catheter near said proximal end proximal to said membrane.

3. A catheter system for endovascular procedures of claim 2 wherein said primary catheter includes an occlusive balloon attached to said primary catheter near said distal end for occluding a vein, one of said plurality of lumen in said primary catheter connected to the interior of said balloon for inflation thereof.

4. A catheter system for endovascular procedures of claim 2 wherein a multi-lumen pulmonary catheter slidably extends through said pulmonary lumen adapted to extend into the pulmonary artery to transport chemical and mechanical agents to clots therein.

5. A catheter system for endovascular procedures of claim 3 wherein a multi-lumen pulmonary catheter slidably extends through said pulmonary lumen adapted to extend into the pulmonary artery to transport chemical and mechanical agents to clots therein.

6. A catheter system for endovascular procedures of claim 1 wherein said primary catheter includes an occlusive balloon attached to said primary catheter near said distal end for occluding a vein, one of said plurality of lumen in said primary catheter connected to the interior of said balloon for inflation thereof, said balloon being distal to said membrane.

7. A catheter system for endovascular procedures of claim 1 wherein at least one of said plurality of lumen terminates in an opening in said primary catheter distal to said membrane, a third multi-lumen catheter slidably extends through said opening in said primary catheter said third catheter adapted to extend into the venous tree to transport chemical and mechanical agents to clots therein.

8. A catheter system for endovascular procedures of claim 2 wherein at least one of said plurality of lumen terminates in an opening in said primary catheter distal to said membrane, a third multi-lumen catheter slidably extends through said opening in said primary catheter said third catheter adapted to extend into the venous tree to transport chemical and mechanical agents to clots therein.

9. A catheter system for endovascular procedures of claim 3 wherein at least one of said plurality of lumen terminates in an opening in said primary catheter distal to said balloon, a third multi-lumen catheter slidably extends through said opening in said primary catheter said third catheter adapted to extend into the venous tree to transport chemical and mechanical agents to clots therein.

10. A catheter system for endovascular procedures comprising a primary catheter for accessing the vascular system from outside the patient, said primary catheter having a plurality of lumen extending from the proximal end through the distal end, an occlusive balloon attached to said primary catheter near said distal end for occluding a vein, one of said plurality of lumen in said primary catheter connected to the interior of said balloon for inflation thereof, a semipermeable membrane surrounding said primary catheter, said membrane forming a pouch having an open interior, said membrane connected to said primary catheter intermediate said balloon and said proximal end, one of said plurality of lumen in said primary catheter adapted to supply oxygen to said interior of said pouch, said semipermeable membrane adapted to pass minute quantities of oxygen to the vascular system, said primary catheter including a pulmonary lumen, said pulmonary lumen extending from said proximal end to an opening in said primary catheter near said proximal end proximal to said membrane, a multi-lumen pulmonary catheter slidably extends through said pulmonary lumen adapted to extend into the pulmonary artery to transport chemical and mechanical agents, at least one of said plurality of lumen terminating in an opening in said primary catheter distal to said balloon, a third multi-lumen catheter slidably extending through said opening in said primary catheter said third catheter adapted to extend into the venous tree to transport chemical and mechanical agents to clots therein whereby venous blood can be oxygenated superior to an embolism.
